(19) United States
(12) Patent Application Publication
(10) Pub. No.: US 2012/0215298 A1
(43) Pub. Date: Aug. 23, 2012

(54) PROSTHESIS AND METHOD OF MANUFACTURING THE SAME

(75) Inventor: Palle M. Hansen, Bjaeverskov (DK)

(73) Assignee: Cook Medical Technologies LLC, Bloomington, IN (US)

(21) Appl. No.: 13/369,922

(22) Filed: Feb. 9, 2012

(30) Foreign Application Priority Data
Feb. 18, 2011 (GB) 1102859.4

(51) Int. Cl. A61F 2/06; B23P 11/00; A61F 2/82

(52) U.S. Cl. 623/1.13; 623/1.16; 29/428

(57) ABSTRACT

A prosthesis (110) comprises a plurality of stent members which are formed of strut elements (120) connected by apexes, wherein at least the proximal apexes of the first stent member or the distal apexes of the second stent member have a shaped portion which is flat or concave. The proximal apexes (122) of a first stent member (116a) are arranged to engage the distal apexes (124) of an adjacent second stent member (116b) when the prosthesis is subjected to longitudinal compression forces. The shaped portion of the apexes prevents interdigitation of the stent members, which could lead to lateral deflection of the prosthesis.

### TECHNICAL FIELD

The present invention relates to a generally tubular prosthesis, in particular to a prosthesis comprising a plurality of stents, to a method of manufacturing such a prosthesis, and to a method of deploying such a prosthesis.

BACKGROUND ART

Prostheses have been used for many years to treat a number of vascular medical conditions. Prostheses in common use comprise at least one stent and are either of the self-expandable type, made for example of a shape memory material, or separately expandable, such as by balloon expansion.

Such prostheses are designed to fit within the vasculature of the patient and need to be appropriate for the size and shape of the lumen. For this purpose, they must be conformable and must not be of a nature that they can apply against the vessel walls forces which could damage or adversely affect the functionality of the vessel or of other organs nearby.

Preferably, such prostheses have a small cross-sectional diameter and/or profile for introducing the prosthesis into the affected body lumen. A configuration which is extremely suited for implantation in a body lumen is a generally cylindrical prosthesis which can radially expand from a first, small, collapsed diameter to a second, larger, expanded diameter. Such prostheses can be deployed in a body lumen at the desired location by means of an introducer assembly, whereby the prosthesis is placed on a catheter and transported through the lumen to the desired location. The prosthesis is retained in a compressed condition by a sheath. Once the introducer assembly has been transported to the desired deployment position the sheath is withdrawn, thus releasing the prosthesis so that it can expand radially into an extended condition. In this regard, the prosthesis may be self-expanding or the catheter may be provided with a balloon or another expansion mechanism which exerts a radial outward pressure on the prosthesis so that the prosthesis expands to a larger diameter.

Lateral deflection in such prostheses can occur during deployment, such as when the prosthesis is longitudinally compressed as it is maneuvered into the desired location in a patient's lumen. Such lateral deflection can be problematic during deployment of the prosthesis. For example, this can distort the sheath to cause increased friction between the sheath and the lumen wall, thus hindering deployment. This may particularly be the case when the prosthesis is to be deployed in a curved lumen, such as the aortic arch, and in extreme cases lateral deflection can prevent the prosthesis from being located in the desired position. Further, such lateral deflection can be dangerous for the patient in more delicate applications, such as in smaller and more delicate vessels including, for instance, cerebral vessels.

Prostheses with various stent arrangements are disclosed in U.S. Pat. No. 7,637,935, U.S. 2009/0204201, WO 98/20810, U.S. Pat. No. 5,776,181, U.S. 2005/0149168, U.S. 2007/0244543, U.S. Pat. No. 7,534,258 and U.S. Pat. No. 6,656,220.

### DISCLOSURE OF THE INVENTION

Aspects of the present invention seek to provide an improved prosthesis in which the aforementioned problems associated with lateral deflection are substantially reduced or overcome.

According to a first aspect of the present invention, there is provided a generally tubular prosthesis comprising a plurality of stent members positioned longitudinally along the prosthesis, each stent member comprising a plurality of strut elements connected by apexes, the prosthesis being arranged such that the proximal apexes of a first stent member engage the distal apexes of an adjacent second stent member when the prosthesis is subjected to longitudinal compression forces, wherein at least the proximal apexes of the first stent member or the distal apexes of the second stent member have a shaped portion which is flat or concave.

The shaped portion of the apexes advantageously prevents interdigitation of adjacent stent members when the prosthesis is subjected to longitudinal compression forces.

In a preferred embodiment the shaped portions are substantially planar, and may be substantially perpendicular to the longitudinal direction of the prosthesis.

Advantageously, both the proximal apexes of the first stent member and the distal apexes of the second stent member may have a shaped portion which is flat or concave. The shaped portions may be substantially planar, and the proximal apexes of the first stent member may be substantially parallel to the distal apexes of the second stent member.

The strut elements may be substantially straight, and may be substantially parallel when the prosthesis is in a compressed configuration.

In an embodiment, each stent member is spaced apart from an adjacent stent member by at least one connector.

Alternatively, or in addition, the stent members may be fastened to a graft material to form a stent graft. The stent members may be fastened to the graft such that each stent member is spaced apart from an adjacent stent member.

Advantageously, the proximal apexes of the first stent member are arranged to engage the distal apexes of the adjacent second stent member when the longitudinal compression forces are of the magnitude experienced during deployment of the prosthesis.

According to a second aspect of the present invention, there is provided a method of manufacturing a generally tubular prosthesis, the method comprising the steps of: forming a plurality of stent members positioned longitudinally along the prosthesis, wherein each stent member comprises a plurality of strut elements connected by apexes; arranging the prosthesis such that the proximal apexes of a first stent member engage the distal apexes of an adjacent second stent member when the prosthesis is subjected to longitudinal compression forces; and forming at least the proximal apexes of the first stent member or the distal apexes of the second stent member to have a shaped portion which is flat or concave.

According to a third aspect of the present invention, there is provided a method of deploying a generally tubular prosthesis comprising a plurality of stent members positioned longitudinally along the prosthesis, each stent member comprising a plurality of strut elements connected by apexes, the prosthesis being arranged such that the proximal apexes of a first stent member engage the distal apexes of an adjacent second stent member when the prosthesis is subjected to longitudinal compression forces, wherein at least the proximal apexes of the first stent member or the distal apexes of the second stent member have a shaped portion which is flat or concave, the method comprising the step of moving the prosthesis longitudinally through a body lumen of a patient during which the proximal apexes of the first stent member engage or come into engagement with the distal apexes of the second stent member, and longitudinal forces are transmitted via the engaged apexes during further longitudinal movement.

### BRIEF DESCRIPTION OF THE DRAWINGS

Preferred embodiments of the invention will now be described, by way of example only, with reference to the accompanying drawings of which:

FIG. 1 shows a developed view of a portion of a prior art prosthesis;

FIG. 2 shows a detailed portion of the prosthesis of FIG. 1;

FIG. 3 shows detail of the prosthesis of FIG. 1 in schematic form, when the prosthesis is experiencing longitudinal compression;

FIG. 4 shows an arrangement of a portion of a prosthesis in accordance with a preferred embodiment of the present invention;

FIG. 5 shows a detailed portion of the prosthesis of FIG. 4; and

FIGS. 6 to 8 show modified apex portions of prostheses in accordance with the present invention.

### DESCRIPTION OF THE PREFERRED EMBODIMENTS

For the purposes of this disclosure, the term “prosthesis” means any replacement for a body part or function of that body part. It can also be used to refer to a device that enhances or adds functionality to a physiological system.

The term “stent” or “stent member” means any device or structure that adds rigidity, expansion force or support to a prosthesis or body lumen.

The term “stent graft” refers to a prosthesis comprising a graft material that forms a lumen through at least a portion of its length and has a number of stent members attached thereto.

Further, when used in connection with description of a stent, stent graft or other implantable medical device or prosthesis, the term “proximal” refers to a part or position closest to the patient's heart, that is upstream in the direction of blood flow, when the prosthesis is in situ, while the term “distal” refers to a part or position furthest from the heart.

In general, prostheses according to the present invention may comprise a plurality of longitudinally spaced apart stents. Each stent may be formed from a resilient wire such as Nitinol wire and may be of generally zig-zag shape, comprising a plurality of struts with a bend between each pair of struts.

Nitinol is a shape memory metal formed from a nickel-titanium (NiTi) alloy that “remembers” its geometry. The wire may be formed into the desired zig-zag shape and then heat treated to retain that shape. After cooling, if it is deformed, it will return to the desired zig-zag shape. Thus, stents formed from such material are able to be radially compressed, for example so as to allow their deployment, and will return to a desired expanded form once the compression forces have been removed.

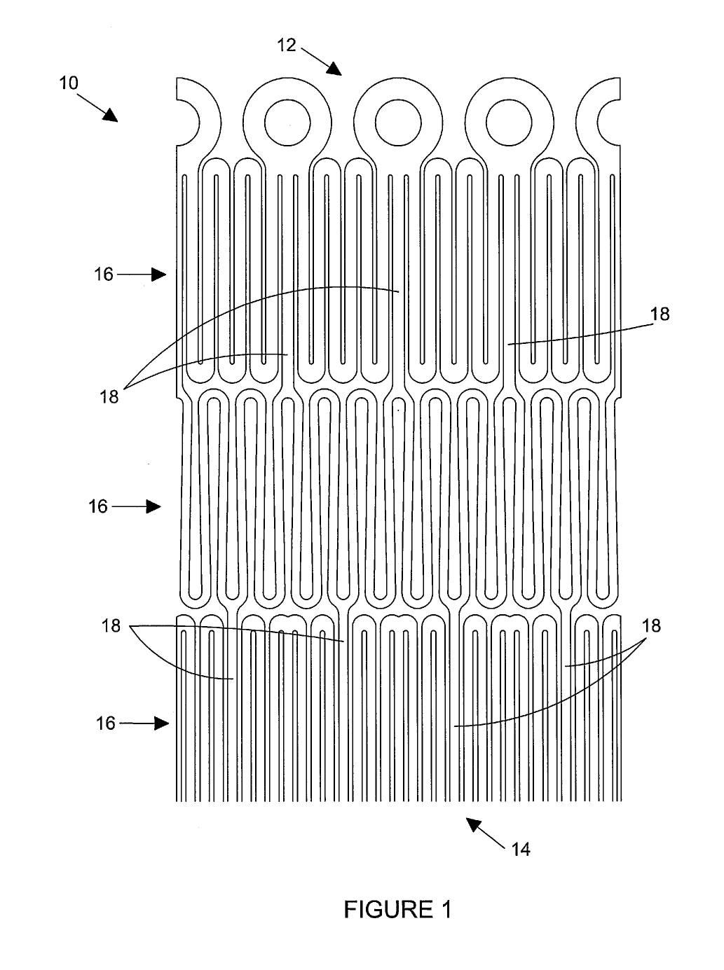

Referring now to FIG. 1 of the accompanying drawings, a portion of a prior art prosthesis 10 is shown. The prosthesis 10 comprises a distal end 12 and a proximal end 14, with a plurality of stent members or stents 16 spaced apart along the length of the prosthesis 10. Each stent 16 is spaced apart from the adjacent stent(s) 16 in the longitudinal direction of the prosthesis 10 by connectors 18. The connectors 18 are flexible enough to allow for curvature of the prosthesis 10 during deployment.

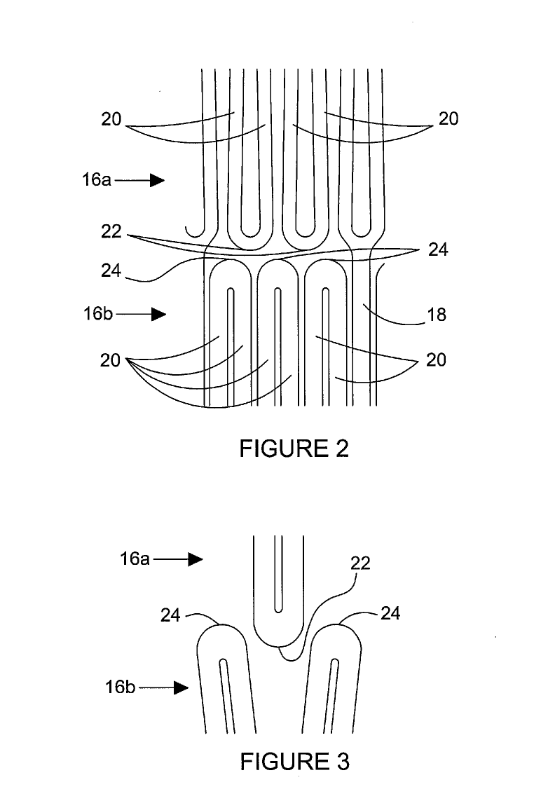

As can be seen from FIG. 2, each stent 16 is formed from a plurality of struts 20 with a bend between each pair of struts 20. The bends form apexes, and the stents 16 are arranged such that the proximal apexes 22 of one stent 16a are facing the distal apexes 24 of the adjacent stent 16b. Adjacent stents 16a, 16b are in a staggered arrangement in which the proximal apexes 22 of one stent 16a are laterally offset from the distal apexes 24 of the adjacent stent 16b. The apexes are of generally rounded form.

Only two stents 16a and 16b are shown in FIG. 2, although this is for clarity purposes only and it will be understood that the prosthesis 10 may in reality comprise more stents 16 spaced apart along the length of the prosthesis 10.

The prior art prosthesis 10 shown in FIG. 2 is in the compressed configuration. The prosthesis 10 will be in this configuration during its deployment in a lumen, and will in practice be held in the compressed configuration by means of a sheath (not shown) which forms a part of the introducer assembly.

Once the introducer assembly is in the desired deployment position, the sheath is withdrawn, thus releasing the prosthesis 10 so that it can expand radially into an expanded condition. The sheath can then be withdrawn completely from the patient.

During deployment of the prosthesis 10, the compressed prosthesis 10 will be pushed longitudinally through the patient's vasculature by the introducer assembly. The prosthesis 10 will be subjected to longitudinal compression forces, and will thus experience longitudinal compression. When the prosthesis 10 is subjected to such longitudinal compression forces, the proximal apexes 22 of the stent 16a move towards the distal apexes 24 of the adjacent stent 16b such that the space between adjacent stents diminishes.

The connectors 18 must be flexible enough to allow for curvature of the prosthesis 10 during deployment. As such, the connectors are not stiff enough to prevent engagement of adjacent stents 16a, 16b.

Continued longitudinal compression of the prosthesis 10, to the magnitude of that experienced during deployment, causes adjacent stents 16a, 16b to firstly engage and then to interdigitate or wedge. That is to say, the proximal apexes 22 of one stent 16a will push past the distal apexes 24 of the adjacent stent 16b such that the struts 20 lie next to each other in the radial direction and the stents 16a, 16b overlap. The rounded form of the proximal 22 and distal 24 apexes allows the apexes to push past each other and interdigitate.

FIG. 3 shows, schematically, a portion of the prosthesis 10 in the compressed configuration, under the influence of longitudinal compression forces. The stents 16a, 16b are beginning to interdigitate as described above. Such interdigitation causes lateral deflection of the prosthesis 10. It will be understood that this lateral deflection will be magnified if interdigitation occurs throughout the prosthesis 10.

Such lateral deflection of the prosthesis 10 during deployment thereof is undesirable, as it can cause the prosthesis 10 to press upon the inner surface of the sheath to cause increased friction between the outer surface of the sheath and the lumen wall, thus hindering deployment. This may particularly be the case when the prosthesis 10 is to be deployed in a curved lumen, such as the aortic arch, and in extreme cases lateral deflection can prevent the stent from being located in the desired position. Further, such lateral deflection can be dangerous for the patient in more delicate applications, such as in smaller and more delicate vessels including, for instance, cerebral vessels.

This lateral deflection may be compounded by the action of the connectors 18 when subjected to longitudinal compression. In this regard, the connectors 18 deform under longitudinal compression and this deformation could cause at least a portion of the connectors 18 to press upon the inner surface of the sheath to further increase the friction between the outer surface of the sheath and the lumen wall, thus further hindering deployment.

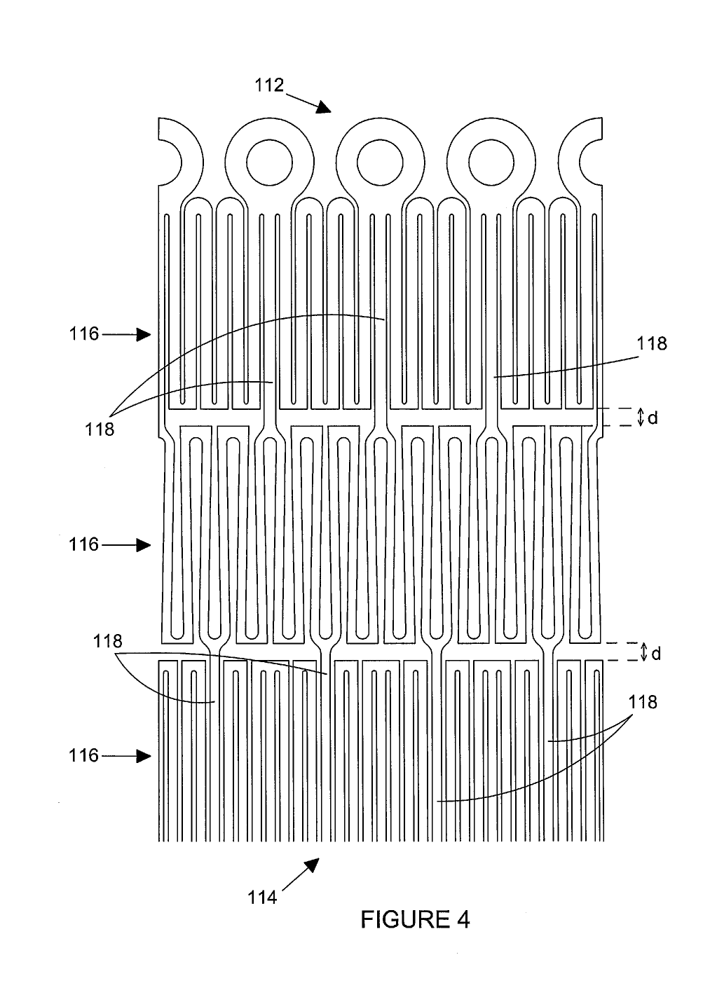

FIG. 4 shows a portion of a prosthesis 110 according to a preferred embodiment of the present invention. As with the prior art prosthesis shown in FIGS. 1 to 3, the prosthesis 110 comprises a distal end 112 and a proximal end 114, with a plurality of stents 116 spaced apart along the length of the prosthesis 110. Each stent 116 is spaced apart from the adjacent stent(s) 116 in the longitudinal direction of the prosthesis 110 by connectors 118. The connectors 118 are flexible enough to allow for curvature of the prosthesis 110 during deployment. Each stent 116 is spaced apart from the adjacent stent(s) 116 in the longitudinal direction of the prosthesis 110 by a distance “d”. In this embodiment, “d” is 0.1 mm. The longitudinal length of the stents 116 in this embodiment is 2 mm. As such, the distance “d” is 5% of the longitudinal length of the stents 116.

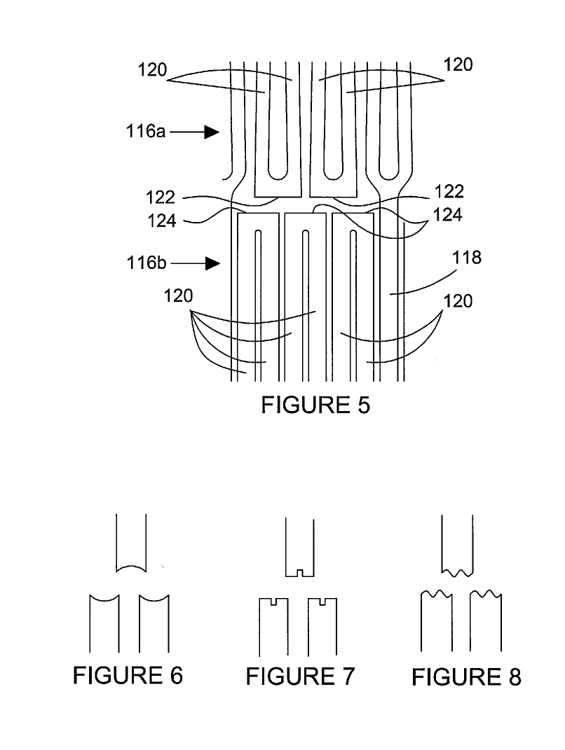

As can be seen from FIG. 5, each stent 116 is formed into a generally zig-zag shape, and comprises a plurality of struts 120 with an apex 122, 124 between each pair of struts 120. The struts 120 are substantially straight. The apexes 122, 124 have a generally flat or flattened shape, that is to say that, instead of forming a generally rounded bend, the apexes between pairs of struts 120 form a more flattened, planar shape. In the present embodiment, the connection between pairs of struts 120 is substantially straight. The stents 116 are arranged such that the proximal apexes 122 of one stent 116a are facing the distal apexes 124 of the adjacent stent 116b, and the shaped or flat portions of the proximal apexes 122 and distal apexes 124 are substantially parallel. Further, the shaped or flat portions of the proximal apexes 122 and distal apexes 124 are substantially perpendicular to the longitudinal direction of the prosthesis 110.

Adjacent stents 116a, 116b are in a staggered arrangement in which the proximal apexes 122 of one stent 116a are laterally offset from the distal apexes 124 of the adjacent stent 116b.

FIG. 5 shows a detailed portion of the prosthesis 110 in the compressed configuration. When in this compressed configuration, each strut 120 lies substantially parallel in side-by-side relation to each other struts 120 of the same stent 116. The prosthesis 110 is in the compressed configuration during its deployment in a lumen, and is in practice held in the compressed is configuration by means of a sheath (not shown) which forms a part of the introducer assembly.

During deployment, the compressed prosthesis 110 is pushed longitudinally through the patient's vasculature by the introducer assembly. The prosthesis 110 is subjected to longitudinal compression forces, and thus experiences longitudinal compression. The flat shape of the proximal 122 and distal 124 apexes provides respective abutment surfaces for adjacent stents 116a, 116b, such that when the prosthesis 110 is subjected to such longitudinal compression forces the flat and parallel surfaces of the proximal apexes 122 of the stent 116a engage and abut the flat and parallel surfaces of the distal apexes 124 of the adjacent stent 116b.

Engagement of the shaped or flat proximal apexes 122 of one stent 116a with the shaped or flat distal apexes 124 of the adjacent stent 116b also acts to prevent excessive deformation of the connectors 118.

An advantage of the above described arrangement is therefore that the proximal apexes 122 of one stent 116a are prevented from pushing past the distal apexes 124 of the adjacent stent 116b. Thus, interdigitation of the struts 120 is avoided. The avoidance of interdigitation in this way leads in turn to a prevention of lateral deflection of the prosthesis 110 during longitudinal compression, for example during deployment of the prosthesis 110. The integrity of the prosthesis 110 shape is therefore maintained during deployment.

Another advantage of the above described arrangement is that excessive deformation of the connectors 118 is prevented. Deformation of the connectors could in itself lead to lateral deflection of the prosthesis 110.

An advantage of the above described arrangement is that the effect of the prosthesis 110 upon the sheath during deployment is substantially reduced or eliminated. As a result, friction between the sheath and the lumen wall, which can be problematic and/or dangerous during deployment of the prosthesis 110, is substantially reduced or eliminated.

In a modified arrangement, the stents may be formed from any suitable shape memory alloy. Alternatively, the stents may be formed from a metal such as stainless steel, or a suitable plastics material.

In a further modified arrangement, the stents may be stitched or otherwise fastened onto a tubular graft material, thus forming a stent graft. In this arrangement, connectors are not required to position the stents longitudinally along the prosthesis.

Further, although in the preferred embodiment the stents are spaced apart by a distance “d” of 0.1 mm, in modified arrangements the spacing “d” may be at least zero and may be 0.2 mm or less, and is preferably 0.1 mm or less. As such, the stents could be in contact even when the prosthesis is not subjected to longitudinal compression forces.

In addition, the longitudinal length of the stents is 2 mm in the preferred embodiment, however the stent length may be at least 1 mm, preferably at least 1.4 mm, and may be 3 mm or less, preferably 2.5 mm or less.

Although the shaped apexes in the preferred embodiment are flat, in modifications the shaped apexes could take on another form. The shaped portion may comprise the whole of, or a part of, the apex. The apexes are shaped such that the apexes of one stent engage or abut the apexes of an adjacent stent, and are thus prevented from pushing past the apexes of the adjacent stent, when the prosthesis is subjected to longitudinal compression forces. Examples of alternative forms of shaped apexes are concave (see FIG. 6), castellated (see FIG. 7) and undulating (see FIG. 8). In these alternate forms, at least a portion of the apex is flat or concave.

Further, although in the preferred embodiment both the proximal apexes of the first stent and the distal apexes of the second stent have flat or concave shaped portions, only one of these sets of apexes may be so shaped with the other set of apexes being convex as is shown in the prior art arrangement of FIGS. 1 to 4.

In addition, although the apexes of adjacent stents are shown as staggered in the preferred embodiment of FIGS. 4 and 5 and in the alternative embodiments shown in FIGS. 6 to 8, in a modification the apexes of one stent may be in line with the apexes of the adjacent stent in the longitudinal direction.

In a further modified arrangement, the stents could spaced apart from the neighbouring stent(s) by a spacing “d” which is of the order of between zero and 20% of the longitudinal length of the stents. It will be appreciated that a zero spacing corresponds to the apexes being in contact with each other even before longitudinal compression occurs.

The features of the various embodiments described above and their modifications may be substituted for or combined with one another as desired. It is also to be understood that although the claims have single dependencies, the claimed features particularly of the dependent claims can be combined with one another as they would in multiply dependent form.

What is claimed is:

1. A generally tubular prosthesis comprising a plurality of stent members positioned longitudinally along the prosthesis, each stent member comprising a plurality of strut elements connected by apexes, the prosthesis being arranged such that the proximal apexes of a first stent member engage the distal apexes of a longitudinally adjacent second stent member when the prosthesis is subjected to longitudinal compression forces, each of the proximal apexes of the first stent member and the distal apexes of the second stent member having a shaped portion which is flat or concave and having a width, so as to present a line of engagement surfaces to the distal apexes of the second stent member and the proximal apexes of the first stent member respectively, wherein the gaps between circumferentially adjacent engagement surfaces are substantially narrower than the widths of the shaped portions when the prosthesis is in a compressed configuration.

2. A prosthesis according to claim 1, wherein the shaped portions are substantially planar.

3. A prosthesis according to claim 2, wherein the shaped portions are substantially perpendicular to the longitudinal direction of the prosthesis.

4. A prosthesis according to claim 2, wherein the proximal apexes of the first stent member are substantially parallel to the distal apexes of the second stent member.

5. A prosthesis according to claim 1, wherein the strut elements are substantially parallel when the prosthesis is in a compressed configuration.

6. A prosthesis according to claim 1, wherein each stent member is spaced apart from an adjacent stent member by at least one connector.

7. A prosthesis according to claim 1, wherein the stent members are fastened to a graft material to form a stent graft.

8. A prosthesis according to claim 7, wherein the stent members are fastened to the graft such that each stent member is spaced apart from an adjacent stent member.

9. A prosthesis according to claim 1, wherein the proximal apexes of the first stent member are arranged to engage the distal apexes of the adjacent second stent member when the longitudinal compression forces are of a predetermined magnitude.

10. A method of manufacturing a generally tubular prosthesis, the method comprising the steps of:
forming a plurality of stent members positioned longitudinally along the prosthesis, wherein each stent member comprises a plurality of strut elements connected by apexes;
arranging the prosthesis such that the proximal apexes of a first stent member engage the distal apexes of a longitudinally adjacent second stent member when the prosthesis is subjected to longitudinal compression forces; and
forming each of the proximal apexes of the first stent member and the distal apexes of the second stent member to have a shaped portion which is flat or concave and to have a width, so as to present a line of engagement surfaces to the distal apexes of the second stent member and the proximal apexes of the first stent member respectively, wherein the gaps between circumferentially adjacent engagement surfaces are substantially narrower than the widths of the shaped portions when the prosthesis is in a compressed configuration.

11. A method according to claim 10, wherein the shaped portions are formed to be substantially planar.

12. A method according to claim 11, wherein the shaped portions are formed to be substantially perpendicular to the longitudinal direction of the prosthesis.

13. A method according to claim 11, wherein the proximal apexes of the first stent member are formed to be substantially parallel to the distal apexes of the second stent member.

14. A method according to claim 10, wherein the stent members are formed such that the strut elements are substantially parallel when the prosthesis is in a compressed configuration.

15. A method according to claim 10, wherein the prosthesis is formed such that each stent member is spaced apart from an adjacent stent member by at least one connector.

16. A method according to claim 10, wherein the prosthesis is formed into a stent graft in which the stent members are fastened to a graft material.

17. A method according to claim 10, wherein the connectors are arranged such that the proximal apexes of the first stent member are arranged to engage the distal apexes of the adjacent second stent member when the longitudinal compression forces are of a predetermined magnitude.
